(19) United States
(12) Patent Application Publication
(10) Pub. No.: US 2010/0041744 A1
(43) Pub. Date: Feb. 18, 2010

(54) OILY PACLITAXEL COMPOSITION AND FORMULATION FOR CHEMOEMBOLIZATION AND PREPARATION METHOD THEREOF

(75) Inventors: Hesson Chung, Incheon (KR); Seo Young Jeong, Koyang-Si (KR); Ick Chan Kwon, Seoul (KR); Yeong Taek Park, Ansan-Si (KR); In Hyun Lee, Seoul (KR); Jae Hyung Park, Seoul (KR); Jin Wook Chung, Seoul (KR); Young Man Kim, Seoul (KR)

Correspondence Address: BUCHANAN, INGERSOLL & ROONEY PC, POST OFFICE BOX 1404, ALEXANDRIA, VA, 22313-1404, US

(73) Assignee: Daehwa Pharm. Co., Ltd., Shiheung-Si (KR)

(21) Appl. No.: 12/540,207

(22) Filed: Aug. 12, 2009

Related U.S. Application Data
(63) Continuation of application No. 10/489,245, filed on Mar. 11, 2004.

(30) Foreign Application Priority Data
Sep. 13, 2001 (KR) 10-2001-0056538
Jul. 20, 2002 (KR) 10-2002-0042795

(51) Int. Cl. A61K 31/337; A61P 35/00

(52) U.S. Cl. 514/449

(57) ABSTRACT

Oily paclitaxel composition and formulation for chemoembolization and preparation method thereof solubilizing paclitaxel in an oily contrast medium. The composition of the present invention solubilizes paclitaxel and has an advantage of delivering anticancer drug to the target cells by chemoembolization since it is possible to visualize the blood vessel during the chemoembolization process. The present invention also relates to oily paclitaxel composition and formulation additionally comprising chemicals that prevent paclitaxel precipitation for prolonged preservation and the preparation method thereof. Since the composition of the present invention solubilize paclitaxel effectively and can be visualized during chemoembolization, it can be used for TACE to treat hepatoma and other solid tumors.

### TECHNICAL FIELD

The present invention relates to oily paclitaxel composition and formulation for transcatheter arterial chemoembolization (TACE) by solubilizing paclitaxel and the preparation method thereof. The present invention also relates to oily paclitaxel composition and formulation additionally comprising chemicals that prevent paclitaxel precipitation for prolonged preservation and the preparation method thereof.

### BACKGROUND ART

TACE is a cancer treatment method that prevents the nutrition supplies to the cancer tissue by injecting embolizing materials and anticancer agents though the feeding artery of tumor while visualizing the operation process with contrast medium. Since the composition of the present invention solubilizes paclitaxel effectively, it can be used for TACE to treat hepatoma and other solid tumors.

The most widely used TACE is transcatheter arterial chemoembolization through hepatic artery for the treatment of hepatoma. The contrast medium serves as a visualization tool during and after the operation and also causes embolism in the tumor. The anticancer drugs such as doxorubicin (adriamycin), cisplatin and carboplatin are dissolved or suspended in oily contrast medium.

One of the most frequently used contrast media in TACE is iodized oils such as Lipiodol®. The suspension system comprising Lipiodol and above-mentioned anticancer drugs, however, is physically unstable and therefore has many limitations during the operation. The anticancer agents such as doxorubicin and epirubicin are used conventionally for the treatment of hepatoma in Radiology. Most of the anticancer agents, however, are water-soluble materials. Therefore, suspension type formulation, rather than oily solution, was used in TACE (Yoshihiro Katagiri et al., Cancer Chemother. Pharmacol 1989, 23, 238-242). The suspension type formulation, however, cannot be stored for a prolonged period of time since particles aggregate upon storage.

To overcome this stability problem, the anticancer drug is dissolved in the aqueous contrast medium before dispersing the aqueous phase in the oily contrast medium such as Lipiodol®. In other words, the anticancer drug is dissolved in the aqueous contrast medium and mixed with oily contrast medium by pumping method just before administering to a patient. To maximize the stability of the emulsion-, aqueous contrast media such as Urografin (specific gravity 1.328-1.332) or lopamiro (specific gravity 1.17-1.41) are used since they have similar specific gravities with Lipiodol (1.275-1.290) (Takashi Kanematsu et al., Journal of surgical oncology 1984, 25, 218-226, Takafumi Ichida et al., Cancer Chemother. Pharmacol 1994, 33, 74-78). However, only a transient emulsion that phase-separates in a few minutes after preparation is produced by the above method. Unstable emulsion system does not provide enough embolizing effect. In reality, phase separation can be observed inside the catheter during the operation. When this unstable emulsion is administered, adriamycin is absorbed immediately to the tissue and therefore does not provide an effect of sustained delivery of anticancer drug.

One of the ideal hepatoma treatments uses a synthetic polymeric anticancer agent, poly(styrene-co-maleic acid)-conjugated neocarzinostatin (SMANCS). SMANCS can be solubilized in Lipiodol since it has both hydrophilic and hydrophobic properties (Konno, T. and Maeda, H., Targetting chemotherapy of hepatocellular carcinoma. Neoplasms of the liver, Eds. Okuda, K., and Ishak, K. G., Springger-Verlag, Berlin, P 343-352). Even though SMANCS/Lipiodol formulation has solved the stability problems of adriamycin/Lipiodol formulation, SMANCS/Lipiodol formulation is not widely used due to the high price and severe toxic side effects.

On the other hand, paclitaxel, an anticancer agent, shows excellent cytotoxicity to ovarian cancer, breast cancer, esophagus cancer, melanoma and leukemia. Paclitaxel has been commerciallized as intravenous injection Taxol® by Bristol-Myers Squibb Company.

Paclitaxel is one of the water-insoluble drug and therefore the solubilization technique has been developed along with the drug itself. One of the examples in the solubilization technique is the use of solubilizing agent for systemic administration such as intravenous injection. The above-mentioned Taxol® uses Cremophor EL (polyoxyethylene 35 castor oil) and ethanol as solubilizing agents. Taxol® is a pre-concentrate type emulsion formulation that forms microemulsion spontaneously when dispersed in excess amount of water (U.S. Pat. No. 5,438,072). It is known, however, that solubilizing agent in Taxoli causes toxic side effects. Therefore, many studies are performed to develop new paclitaxel formulations with high anticancer activity and low toxic effects.

### SUMMARY OF THE INVENTION

The object of the present invention is to use paclitaxel in transcatheter arterial chemoembolization by solubilizing paclitaxel.

Therefore, one of the objects of the present invention is to provide a new composition of paclitaxel that can solubilize paclitaxel.

More particularly, the object of the present invention is to provide an oily paclitaxel formulation that can be used for the treatment of solid tumors by transcatheter arterial chemoembolization

Also, another object of the present invention is to provide an oily paclitaxel formulation that can maintain the original composition stably during the transcatheter arterial chemoembolization process.

Another object of the present invention is to provide a preparation process of the above composition of paclitaxel.

Another object of the present invention is to provide a paclitaxel composition for transcatheter arterial chemoembolization comprising an additional component to prevent paclitaxel precipitation.

### DETAILED DESCRIPTION OF THE INVENTION

While trying to find a paclitaxel formulation that can be used in transcatheter arterial chemoembolization to meet the above mentioned expectations, the present inventors have found unexpectedly that paclitaxel is soluble in the oily contrast medium to form a homogeneous single phase viscous oily liquid of viscosity ranging 40˜180 centipoises (cP).

Also the paclitaxel/oily contrast medium composition can be stored for a long period of time without changing the composition since it is chemically and physically stable. This paclitaxel/oily contrast medium composition has superior physical properties to the conventional Lipiodol formulations using water-soluble anticancer drugs such as doxorubicin. The paclitaxel/oily contrast medium composition of the present invention has similar physical characteristics to SMANCS/Lipiodol formulation. In contrast to the SMANCS/Lipiodol formulation that is too expensive and has toxic side effects, however, the paclitaxel/lipiodol composition uses two relatively inexpensive raw materials and is very easy to prepare reducing the production cost. Also the obtained formulation is stable upon storage.

The oily paclitaxel formulation of the present invention can maintain the original composition stably during the transcatheter arterial chemoembolization process while the conventional Lipiodol/lopamiro/doxorubicin formulation phase-separated immediately after mixing. Therefore, the paclitaxel/oily contrast medium formulation of the present invention can deliver the anticancer drug in a sustained release fashion to the tumor. Also, the formulation can be stored for a long period of time due to its excellent stability. Moreover, the result described hereinbelow shows that the formulation of the present invention has an excellent embolization effect and anticancer activity when TACE was performed through hepatic artery in an animal model. Therefore, it is expected that the formulation of the present invention can be used in TACE.

Even though the most typical TACE is TACE through hepatic artery, it can be applied to a variety of solid tumors. For instance, SMANCS/Lipiodol formulation has been used for the targeted therapy of renal cancer by performing TACE through renal artery (K. Tsuchiya, Tumor-targeted chemotherapy with SMANCS in Lipiodol for renal cell carcinoma: longer survival with larger size tumors. Urology. 2000 April; 55(4):495-500).

The object of the present invention is to use paclitaxel in transcatheter arterial chemoembolization by solubilizing paclitaxel.

An example of an oily contrast medium that can be used in preparing the paclitaxel/oily contrast medium composition is iodized oil. The iodized oils include iodized poppy seed oil such as Lipiodol (Laboratoire Guerbet, France), Ethiodol (Savage Laboratories, Melville, N.Y.) and iodized soybean oil. The iodized soybean oil is described by Ma Tai (The effect of oral iodized oil on prevention and treatment of endemic goiter. Chinese Med. J. 61 (9):533, 1981).

The iodine content of the iodized oil used as oily contrast medium in the present invention is preferably 30˜50% by weight. More preferably, the iodine content is 35˜45% by weight. It is the most preferable to use Lipiodol as the oily contrast medium.

The amount of paclitaxel in the paclitaxel/oily contrast medium of the present invention is 0.0001˜10 mg per 1 ml of oily contrast medium. When the amount of paclitaxel exceeds 10 mg per 1 ml of oily contrast medium, it is not preferable since the excess paclitaxel precipitates. On the other hand, anticancer activity is too low when the amount of paclitaxel is lower than 0.0001 mg per 1 ml of oily contrast medium.

Also, animal oils such as squalene or vegetable oils such as soybean oil can be included additionally in the paclitaxel/oily contrast medium composition of the present invention. By substituting parts of the oily contrast medium with animal oils, vegetable oils or their mixture, the cost of producing the formulation can be lowered without sacrificing the efficacy or stability. The ratio of oily contrast medium: animal oil and/or vegetable oil is 1:0.01˜1 by volume. More preferably, the above ratio is 1:0.01˜0.5.

The paclitaxel/oily contrast medium composition of the present invention can be easily prepared by adding paclitaxel to the oily contrast medium according to the above composition range and solubilizing paclitaxel by stirring the mixture at room temperature. To speed up the solubilization process, it is acceptable to raise the temperature to 35˜45° C. or to sonicate in a bath type sonicator. The prepared paclitaxel/oily contrast medium composition is stored after sterilization process. It is acceptable to use sterilized raw materials and to mix them under a sterile environment. Or the paclitaxel/oily contrast medium composition can be sterilized by injecting through a sterile syringe filter (pore size 200 μm, PVDF sterile filter). It is also acceptable to sterilize and to mix the oily contrast medium and paclitaxel or to sterilize the composition by using gamma ray or EO gas sterilization protocols.

The paclitaxel/oily contrast medium composition of the present invention prepared as above was stable for more than 60 days at room temperature.

In the above oily composition, paclitaxel is precipitated out of the oily solution eventually even though paclitaxel is stably solubilized for 2 months. The precipitation is formed by inter- and intra-molecular hydrogen bonding between paclitaxel molecules. The present inventors have found that the precipitation can be effectively prevented by adding chemicals that form hydrogen bonding with paclitaxel or that disturb inter- and intra-molecular hydrogen bonding between paclitaxel molecules. The oily paclitaxel composition does not form precipitation after 2 months if the oily contrast medium itself can form hydrogen bonding with paclitaxel.

When Lipiodol, one of the most popularly used oily contrast media, was used, Lipiodol cannot form hydrogen bonding with paclitaxel due to the chemical nature of Lipiodol molecules. In this case, the chemicals which can form hydrogen bonding with paclitaxel in Lipiodol solution can prevent paclitaxel precipitation. For example, paclitaxel precipitation was prevented when tricaprylin was added to the oily paclitaxel composition since the hydrogen bonding between paclitaxel and tricaprylin was formed instead of that between paclitaxel molecules.

The contents of paclitaxel and the oily contrast medium in the oily paclitaxel composition after prolonged storage depend on the preparation process. If the composition was prepared in the absence of moisture or oxygen and also without being heated, the composition is stable for longer period of time since oxidation and hydrolysis of the components can be minimized. The precipitation process, however, is a thermodynamically driven process unlike other destabilization processes. Therefore, precipitation formation is unavoidable for the present oily paclitaxel composition no matter what precaution is taken during and after preparation. The rate of precipitation formation depends on the concentration of paclitaxel in the oily composition. In case paclitaxel concentrations are 10 mg/ml and 5 mg/ml in the oily composition, the precipitation is formed in approximately 60 and 120 days, respectively, at ambient temperatures. Therefore, the oily paclitaxel formulation can be stable for more than 1 year only when additional component that inhibits paclitaxel precipitation is added to the composition.

Therefore, the oily paclitaxel composition of the present invention can additionally comprise a component that inhibits paclitaxel precipitation. The solubility of paclitaxel in the oily composition increases up to 13 mg/ml in this case.

In other words, the amount of paclitaxel in the paclitaxel/oily contrast medium of the present invention is 0.0001˜13 mg, and the amount of the chemical that prevents paclitaxel precipitation is 0.01˜1 ml per 1 ml of oily contrast medium.

An example of the oily contrast medium is the same as described above.

The chemicals that can prevent paclitaxel precipitation in preparing the paclitaxel/oily contrast medium composition include an agent that forms hydrogen bonding with paclitaxel or a chaotropic agent that disturbs hydrogen bonding between paclitaxel molecules.

Chemicals that can form hydrogen bonding with the above paclitaxel molecule include alcohols, polyols, oils, lipids, polymers or peptides. Alcohols include methanol, ethanol, propanol, isopropanol, butanol and fatty alcohols. Polyols include ethylene glycol, propylene glycol and polyethyleneglycol. Oils include triglycerides, diglyceride, monoglycerides, tocopherol and animal or plant oils which are the mixtures of triglycerides, diglyceride, monoglycerides and other minor components. Lipids include phospholipid, neutral lipid, cationic lipid, anionic lipid and fatty acid. Polymers include poly(lactic acid), poly(glycolic acid) and their copolymers, chitosan, alginate, hyaluronate, daxtran and poly(ε-caprolatone). Chaotropic agents include dimethylsulfoxide (DMSO) and amides.

The paclitaxel/oily contrast medium of the present invention was stable for more than 200 days at ambient temperatures when a chemical that prevents paclitaxel precipitation was added.

The paclitaxel/oily contrast medium of the present invention can be used for TACE to treat solid tumors and has a viscosity of 40˜180 cP.

Also the amount and the method of the administration of the paclitaxel/oily contrast medium composition of the present invention can be varied up to the decision of the doctor depending on the age, sex, weight, and severeness of the patient. Generally, TACE can be performed once in 1˜4 months and can be repeated. Two to 15 ml of the formulation is injected through the feeding artery of a solid tumor, for instance through hepatic artery in case of hepatoma.

The invention will be further illustrated by the following examples. It should be understood that these examples are intended to be illustrative only and the present invention is not limited-to the conditions, materials or devices recited therein.

### BRIEF DESCRIPTION OF THE DRAWINGS

FIG. 1 is a computed tomography (CT) picture obtained 1 week after selectively administering 0.3 cc of paclitaxel/lipiodol formulation of the present invention to the rabbit hepatoma by transcatheter arterial chemoembolization. The amount of the administered paclitaxel corresponds to A) 1 mg, B) 3 mg and C) 0 mg.

FIG. 2 is a graph showing the concentration of paclitaxel in the hepatoma and neighboring normal liver tissues one week after selectively administering 0.3 cc of paclitaxel/lipiodol formulation of the present invention to the rabbit hepatoma by transcatheter arterial chemoembolization. The quantitative analysis of paclitaxel was performed by high performance liquid chromatography (HPLC). The amount of the administered paclitaxel corresponds to A) 1 mg and B) 3 mg.

FIG. 3 is a graph showing the percent ratio of the viable tumor in total hepatoma tissue one week after selectively administering 0.3 cc (the groups administered with 1 mg and 3 mg of paclitaxel) and 0.4 cc (the group administered with 4 mg of paclitaxel) of paclitaxel/lipiodol formulation of the present invention to the rabbit hepatoma by transcatheter arterial chemoembolization. In case of the negative control group, 0.3 cc of Lipiodol was administered.

FIG. 4 is a graph showing the concentration of paclitaxel in hepatoma, left lobe and right lobe one week after selectively administering 0.4 cc (the group administered with 4 mg of paclitaxel) of paclitaxel/lipiodol formulation of the present invention to the rabbit hepatoma by transcatheter arterial chemoembolization.
--; concentration of paclitaxel in hepatoma,
-◯-; concentration of paclitaxel in left lobe,
-▾-; concentration of paclitaxel in right lobe.

FIG. 5 is a photograph of paclitaxel/lipiodol and paclitaxel/lipiodol/tricaprylin formulations after 200 days of storage at ambient temperature.
A; photograph of paclitaxel/lipiodol formulation,
B; photograph of paclitaxel/lipiodol formulation under polarized light microscope,
C; photograph of paclitaxel/lipiodol/tricaprylin formulation,
D; photograph of paclitaxel/lipiodol/tricaprylin formulation under polarized light microscope.

FIG. 6 is a graph showing the thickness of mice footpad after injecting 20 μl of paclitaxel/lipiodol/tricaprylin formulation (the group administered with 200 μg of paclitaxel) 5 days after inoculating melanoma cells. In case of the control group, 20 μl of lipiodol/tricaprylin was administered Untreated group was also used as a negative control.
--; group administered with 20 μl of paclitaxel/lipiodol/tricaprylin formulation (200 μg of paclitaxel),.
-◯-; group administered with 20 μl of lipiodol/tricaprylin formulation,
-▾-; untreated group.

FIG. 7 is a graph showing the number of surviving mice after injecting 20 μl of paclitaxel/lipiodol/tricaprylin formulation (the group administered with 200 μg of paclitaxel) 5 days after inoculating melanoma cells. Untreated group was used as a negative control.
--; group administered with 20 μl of paclitaxel/lipiodol/tricaprylin formulation (200 μg of paclitaxel),
-◯-; untreated group.

### EXAMPLES

### Example 1

Preparation of Paclitaxel/Lipiodol Composition

One milliliter of Lipiodol (Lipiodol Ultra-fluid, Laboratoire Guerbet, France, Iodine content 38% by weight) was used as an oily contrast medium. Lipiodol and 2, 4, 6, 8, 10 or 11 mg of paclitaxel (Samyang Genex, Korea) were added in test tubes (micro test tubes with safety lock, polyethylene, 1.5 ml, Eppendorf AG, Germany) and solubilized by stirring at room temperature. To speed up the solubilization process, it is acceptable to raise the temperature to 35˜45° C. or to sonicate in a bath type sonicator. When 2˜10 mg of paclitaxel was added in 1 ml of Lipiodol, paclitaxel was completely solubilized in Lipiodol as evidenced by the formation of clear single liquid phase. When 11 mg of paclitaxel was added to 1 ml of Lipiodol, however, clear liquid was formed initially but the turbidity of the solution increased after overnight storage at room temperature. Paclitaxel precipitation was observed under a microscopy. Therefore, it was confirmed that the solubility of paclitaxel in Lipiodol is approximately 10 mg/ml at room temperature (24˜28° C.). Viscosity of the paclitaxel/lipiodol (10 mg/1 ml) formulation was measured using a Kinematic viscometer Cannon-Fenske Type, Calibrated, Cat. No. 13-617E, Size 200, Fisher Scientific, Pittsburgh, Pa.) by measuring the falling time of the liquid formulation and was 67 cP at 25° C. Since the viscosity was higher than 45 cP, embolization effect is maximized, it is expected that paclitaxel/Lipiodol composition has an excellent embolization effect.

### Example 2

Physical Stability of Paclitaxel/Lipiodol Composition

One milliliter of Lipiodol (Lipiodol Ultra-fluid, Laboratoire Guerbet, France, Iodine content 38% by weight) and 10 mg of paclitaxel (Samyang Genex, Korea) were added in test tubes and solubilized by stirring at room temperature. To speed up the solubilization process, the temperature of the mixture was raised to 40° C. Paclitaxel was completely solubilized in Lipiodol as evidenced by the formation of clear single liquid phase. The prepared composition was sterilized by injecting through a syringe filter (200 μm pore size, PVDF filter) and stored at room temperature and at 4° C. for 60 days to observe the physical stability and the degradation of paclitaxel. There was no change in the color and odor of the formulation. Phase separation or precipitation did not occur. Degradation of paclitaxel was not observed as evidenced by the analysis performed by HPLC.

The HPLC conditions were as follows.
Pump: SP8810 precision isocratic pump (Spectra-Physics Inc., San Jose, Calif.)
Column: Waters Bondpack C18 Column (3.9 mm×300 mm, Waters Corp., Milford, Mass.)
Mobile phase: acetonitrile and water 50% (w/w) each
Flow rate: 1 ml/min
Detector: Spectra 100 variable wavelength (Spectra-Physics)

### Example 3

Physical Stability of Paclitaxel/Ethiodol Composition

Except that Ethiodol (Savage Laboratories, Melville, N.Y.) was used instead of Lipiodol as an oily contrast medium, the oily paclitaxel composition was prepared as described in Example 2. Paclitaxel was completely solubilized in Ethiodol as evidenced by the formation of clear single liquid phase. The physical stability of the prepared composition was tested by the same methods as in Example 2. The prepared composition was sterilized and stored at room temperature and at 4° C. for 60 days to observe the physical stability and the degradation of paclitaxel. There was no change in the color and odor of the formulation. Phase separation or precipitation did not occur. Degradation of paclitaxel was not observed as evidenced by the analysis performed by HPLC.

### Experimental Example 1

Preparation of Hepatoma Animal Model

VX2 tumor provided by Deutsches Krebsforschungszentrum Tumorbank (Germany) was transplanted into the thigh of rabbits (New Zealand White). After 2 weeks, the rabbits having 1˜2 cm tumors were sacrificed by intravenous injection of 10 ml of pentothal sodium solution (62.5 mg/kg). The tumors were excised along with the tissues around them after disinfection with Iodine solution and alcohol, removing the hair and cutting the skin over the tumor site. The tumor was cut to remove the central necrotic portion. The viable peripheral tumor tissue was mixed with calcium and magnesium-free Hank's balanced salt solution (Grand Island Biological Co., Grand Island, N.Y.) and cut into very small pieces with scissors and surgical mess. The tumor solution was mixed with 5 ml of RMPI-1640 (Rosewell Park Memorial Institute, Rosewell Park, N.Y.). The mixture was diluted to 1×10⁶ tumor cells/mm³.

Injection of Tumors Cell Solution into Rabbit Liver

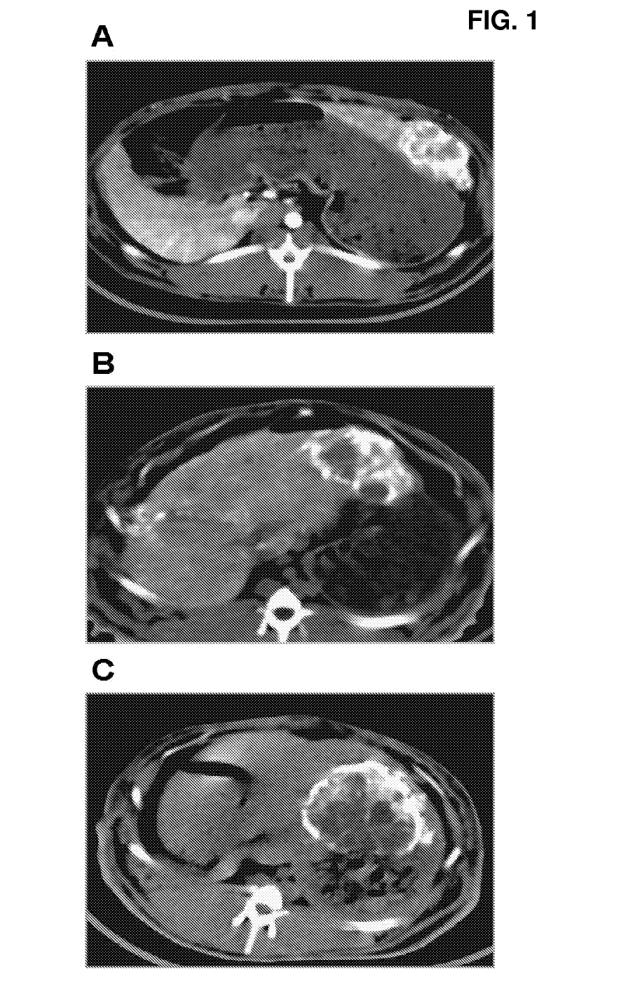

Five hundred milliliters of phosphate buffered saline was administered through the vein of the ear via 23 G needle as a first step. Through this rabbit vein, 40 ml of phosphate buffered saline mixed with 500 mg of pentothal sodium was injected at a flow rate of 1 ml/min to anesthetize a rabbit. The total dose of the solution was 1.5 ml/kg. The hair in the abdomen was removed, and the skin was disinfected with Iodine solution and alcohol. Under the ultrasound guide, 0.1 ml of the tumor tissue solution was injected to the liver parenchyma of the left lobe with a 1 ml syringe through a 22 G needle. The tumor tissue solution was injected to the left lobe among the 5 lobes in the rabbit liver since it is the easiest to observe with the ultrasound (FIG. 1). To prevent secondary infection, antibiotic (PenbrexR, 250 mg) was injected intravenously. After the injection of the tumor tissue solution, the rabbits were grown in a rabbit cage with normal meals. In two weeks after the transplantation of tumor cells, tumor was identified by ultrasound observation and CT. The tumor growth could be roughly predicted by the growth curve. The ultrasound observation was performed every 3 days, and CT was performed every week starting 2 weeks after the transplantation to follow up the position and size of the tumor.

### Example 4

Transcatheter Arterial Chemoembolization with Paclitaxel/Lipiodol Composition in Hepatoma Animal Model

One milliliter of Lipiodol and 3.33 mg or 10 mg each of paclitaxel (Samyang Genex, Korea) were added in test tubes and solubilized by stirring at room temperature. To speed up the solubilization process, the temperature of the mixture was raised to 40° C. Paclitaxel was completely solubilized in Lipiodol as evidenced by the formation of clear single liquid phase. The prepared composition was sterilized by injecting through a syringe filter (200 μm pore size, PVDF filter).

In the hepatoma animal model prepared in Experimental Example 1, TACE was performed through a catheter into the feeding artery of the tumor 0.3 ml of the paclitaxel/Lipiodol formulation of the present invention. Therefore, the dose of paclitaxel corresponds to 1 mg and 3 mg, respectively. As a negative control group, 0.3 cc of Lipiodol was injected to the hepatoma animal model. Lipiodol was taken up selectively into the tumor tissue in one week after the surgery as shown by the computed tomographic picture in FIG. 1.

### Example 5

Analysis of Paclitaxel Concentration in the Hepatoma Tissue After the Transcatheter Arterial Chemoembolization with Paclitaxel/Lipiodol Composition

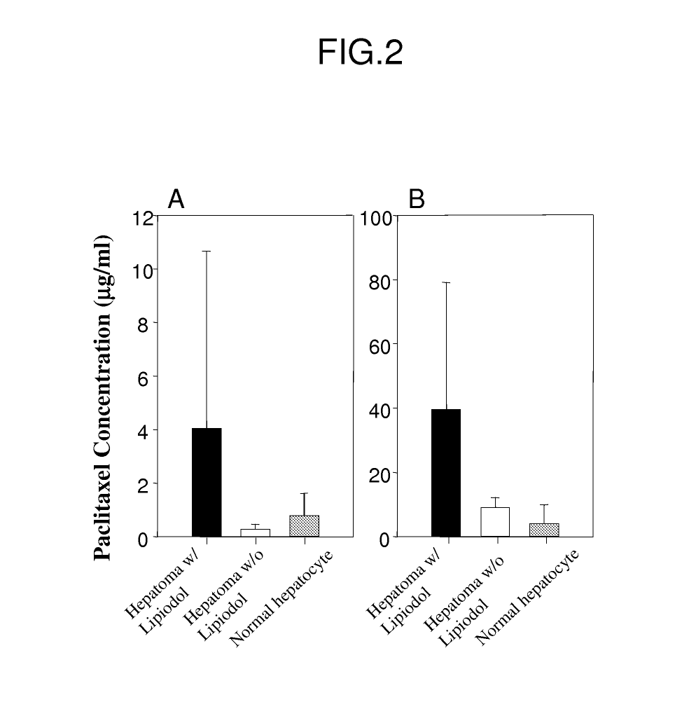

The rabbits were sacrificed in one week after the transcatheter arterial chemoembolization in Example 4, and livers were taken out. The paclitaxel concentration was determined in the tumor tissue that Lipiodol was visually identified, the tumor tissue that Lipiodol is not visually identified and the normal liver tissue neighboring the tumor. Each liver tissue was mixed with a lysis buffer solution [62.5 mM Tris-HCl (pH 6.8), 2% sodium dodecyl sulfate, 5% β-mercaptoethanol, 10% glycerol] and homogenized. After the homogenized mixture was centrifuged, the supernatant was obtained to analyze the paclitaxel concentration by HPLC. The conditions for HPLC were identical to those in Example 2. As explained in Example 4, the paclitaxel concentrations in the liver of the rabbits administered with the formulation corresponding to 1 mg or 3 mg of paclitaxel are shown in FIGS. 2A and 2B, respectively. The concentration of paclitaxel in the hepatoma tissue that Lipiodol was visually identified was the highest. The concentration was relatively high in the hepatoma tissue that Lipiodol was not visually identified. On the other hand, the paclitaxel concentration was negligible in the normal liver tissue neighboring the tumor. Therefore, it was confirmed that paclitaxel distributes selectively in the tumor one week after the operation with the paclitaxel/Lipiodol formulation of the present invention.

### Example 6

Determination of Viable Tumor After the Transcatheter Arterial Chemoembolization with Paclitaxel/Lipiodol Composition

One milliliter of Lipiodol and 3.33 mg or 10 mg each of paclitaxel (Samyang Genex, Korea) were added in test tubes and solubilized by stirring at room temperature. To speed up the solubilization process, the temperature of the mixture was raised to 40° C. Paclitaxel was completely solubilized in Lipiodol as evidenced by the formation of clear single liquid phase. The prepared composition was sterilized by injecting through a syringe filter (200 μm pore size, PVDF filter).

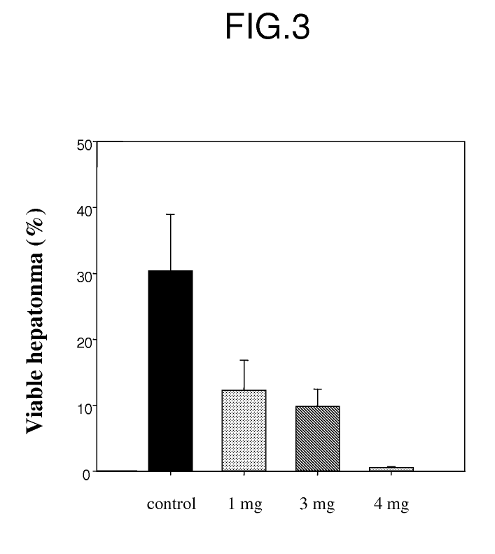

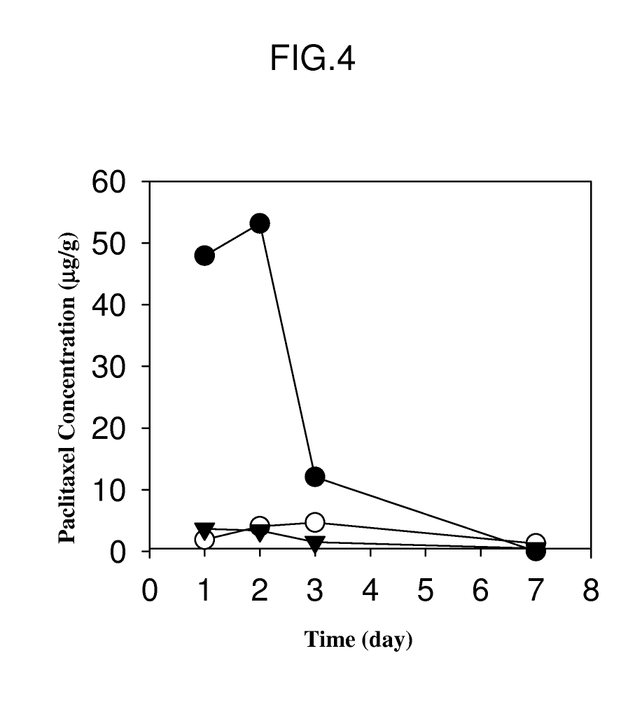

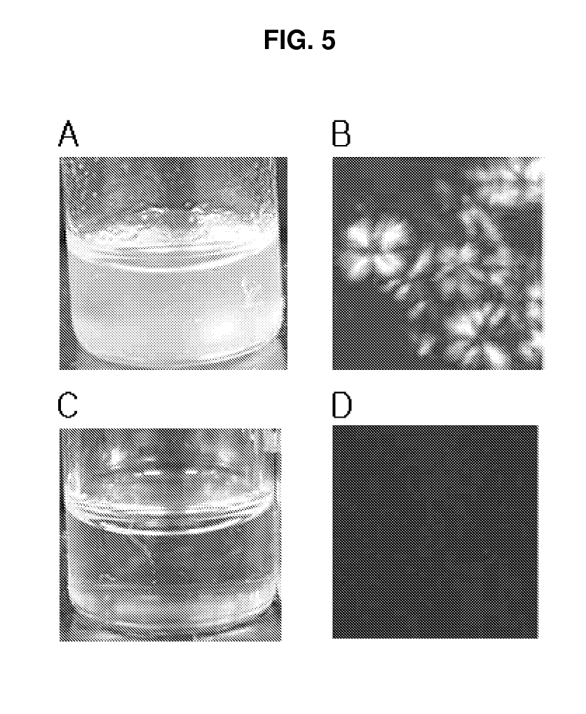

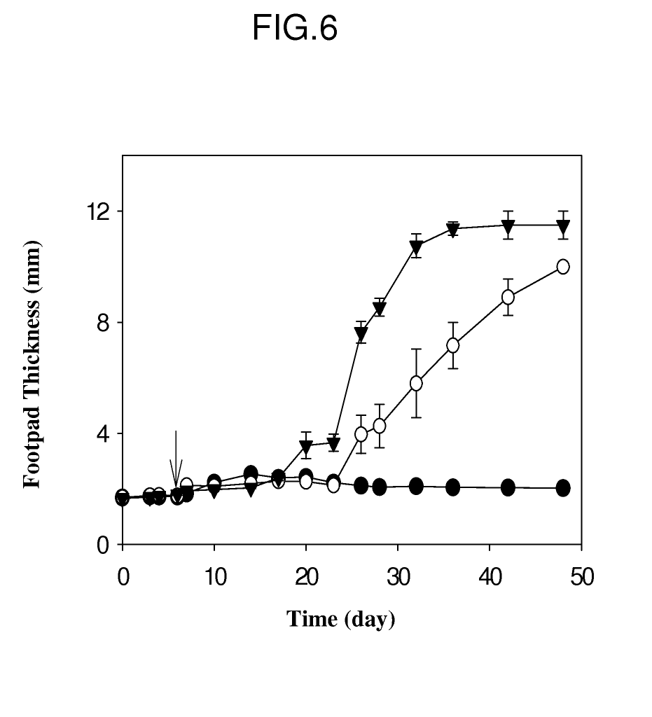

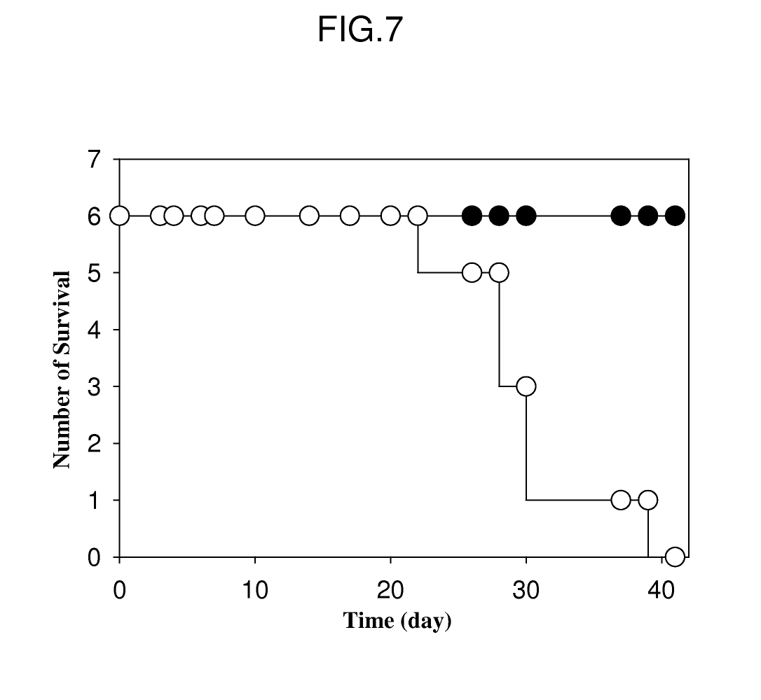

In the hepatoma animal model prepared in Experimental Example 1, TACE was performed through a catheter into the feeding artery of the tumor 0.3 ml (3.33 or 10 mg/ml formulations) or 0.4 ml (10 mg/ml formulation) of the paclitaxel/Lipiodol formulation of the present invention. Therefore, the dose of paclitaxel corresponds to 1 mg, 3 mg or 4 mg, respectively. As a negative control group, 0.3 cc of Lipiodol was injected to the hepatoma animal model. Lipiodol was taken up selectively into the tumor tissue in one week after the surgery as shown by the computed tomographic picture in FIG. 1. The rabbits were sacrificed in one week after the transcatheter arterial chemoembolization, and livers were taken out. The size of the tumors in the groups administered with the paclitaxel/Lipiodol formulations was similar to the negative control group administered with Lipiodol and was 32±5 mm. Pathological examination was performed to distinguish necrotic tumor and viable tumor in the tumor tissue. The viable tumor portion in the total tumor tissue is shown in FIG. 3. In the negative control group, more than 30% of the tumor was viable whereas the viable tumor was 13.2%, 10.4% and 0.6% in the groups of rabbits administered with 1 mg, 3 mg and 4 mg, respectively, of paclitaxel. These result indicate that paclitaxel in the paclitaxel/Lipiodol formulation of the present invention effectively destroys tumor cells.

### Example 7

Preparation of Lipiodol/Soybean Oil/Paclitaxel Composition

One milliliter of Lipiodol, 0.2 ml of soybean oil and 10 mg each of paclitaxel were added in test tubes and solubilized by stirring at room temperature. To speed up the solubilization process, the mixture was sonicated in a bath type sonicator. Paclitaxel was completely solubilized in the mixed oil system of Lipiodol/soybean oil as evidenced by the formation of clear single liquid phase.

### Example 8

Preparation of Lipiodol/Squalene/Paclitaxel Composition

Except that squalene was used instead of soybean oil, and the mixture was heated to 40° C. to speed up the solubilization process, Lipiodol/squalene/paclitaxel composition was prepared by using the same preparation method in Example 6. Paclitaxel was completely solubilized in the mixed oil system of Lipiodol/soybean oil as evidenced by the formation of clear single liquid phase.

### Example 9

Preparation of Paclitaxel/Lipiodol/Tricaprylin Composition and Determination of Its Physical Stability

An oily mixture of 1 ml of Lipiodol (Lipiodol Ultra-fluid, Laboratoire Guerbet, France, Iodine content 38% by weight) and 0.01 ml of tricaprylin (Sigma Chemical Co.) and 10 mg of paclitaxel (Samyang Genex, Korea) were added in a test tube and solubilized by stirring at room temperature. To speed up the solubilization process, the composition was sonicated in a bath type sonicator. Paclitaxel was completely solubilized in the oil mixture of Lipiodol/tricaprylin as evidenced by the formation of clear single liquid phase. The prepared composition was sterilized by injecting through a syringe filter (200 μm pore size, PVDF filter) and stored at room temperature and at 4° C. for 200 days to observe the physical stability and the degradation of paclitaxel. There was no change in the color and odor of the formulation. Phase separation or precipitation did not occur. Degradation of paclitaxel was not observed as evidenced by the analysis performed by HPLC. In case of paclitaxel/lipiodol formulation in Example 1, the composition became turbid due to the precipitation of paclitaxel (FIG. 5A) after 200 days of storage at ambient temperatures. Paclitaxel precipitation was observed under polarized light microscope for paclitaxel/lipiodol composition (FIG. 5B). In contrast, paclitaxel/lipiodol/tricaprylin composition stayed clear (FIG. 5C) without forming paclitaxel precipitation (FIG. 5D). Therefore, the paclitaxel/lipiodol composition can be stabilized for a long period of time by adding tricaprylin as a component to inhibit paclitaxel precipitation.

### Example 10

Preparation of Paclitaxel/Lipiodol/Tricaprylin Composition and Determination of Its Physical Stability

A mixture of 1 ml of Lipiodol (Lipiodol Ultra-fluid, Laboratoire Guerbet, France, Iodine content 38% by weight) and 0.01 ml of tricaprylin (Sigma Chemical Co.) and 12 mg of paclitaxel (Samyang Genex, Korea) were added in a test tube and solubilized by stirring at room temperature. To speed up the solubilization process, the composition was sonicated in a bath type sonicator. Since paclitaxel was completely solubilized in the oil mixture of Lipiodol/tricaprylin as evidenced by the formation of clear single liquid phase, the solubility of paclitaxel is higher in a mixed oil system of lipiodol/tricaprylin than in lipiodol alone.

### Experimental Example 2

Preparation of Melanoma Animal Model

Melanoma cell line, B16F10, spontaneously occurring in C57BL/6J mice was obtained from American Type Culture Collection (ATCC, USA). The cells were cultivated in Dulbeccos Modified Eagle Medium (DMEM, Gibco BRL/Life Technologies, New York, N.Y.), supplemented with 10% fetal bovine serum (FBS, Gibco) and 1% Penicillin/Streptomycin (Gibco). To prepare melanoma animal model, 1×10⁶ cells were dispersed in 100 μl of DMEM and inoculated into rear left footpad of 8-week old C57BL/J mice (Samtaco, Korea).

### Example 11

Determination of Melanoma Size After Injecting Paclitaxel/Lipiodol/Tricaprylin Composition

The paclitaxel/lipiodol/tricaprylin composition prepared in Example 9 was sterilized by injecting through a syringe filter (200 μm pore size, PVDF filter). Twenty microliters of the composition was injected into the inoculation site of rear left footpad 5 days after inoculation of melanoma as in Experimental Example 2. As negative controls, a group injected with 20 μl of lipiodol/tricaprylin (100:1 by volume) and untreated group were used. The size of the melanoma was quantified by measuring the thickness of the footpad and is shown in FIG. 6. Melanoma began to grow 18 and 22 days after inoculation in case of the untreated group and the group treated with lipiodol/tricaprylin, respectively. In contrast, melanoma did not grow at all in the group treated with paclitaxel/lipiodol/tricaprylin proving the marked anticancer activity.

### Example 12

Determination of Survival Time After Injecting Paclitaxel/Lipiodol/Tricaprylin Composition

The paclitaxel/lipiodol/tricaprylin composition prepared in Example 9 was sterilized by injecting through a syringe filter (200 μm pore size, PVDF filter). Twenty microliters of the composition was injected into the inoculation site of rear left footpad 5 days after inoculation as in Experimental Example 2.

Untreated group was used as a negative control. The number of surviving mice is shown in FIG. 7 as a function of time. In-the untreated group, mice began to die 20 days after inoculation. All of the mice died in 48 days after inoculation (n=6). All of the mice treated with paclitaxel/lipiodol/tricaprylin composition stayed healthy and alive showing the marked anticancer activity of the present composition.

### INDUSTRIAL APPLICABILITY

The paclitaxel/oily contrast medium composition of the present invention is a single phase viscous liquid. The composition of the present invention opens up a new administration route for paclitaxel, which has been conventionally administered mainly through intravenous injection. The composition of the present invention can be used for the treatment of hepatoma by transcatheter arterial chemoembolization. The paclitaxel/Lipiodol formulation of the present invention is easy to prepare and to sterilize and is physically and chemically more stable than conventional doxorubicin/Lipiodol formulation. Therefore, the composition is stable during and after the TACE for the treatment of solid tumors, and is stable for at least 60 days at room temperature. Also, the solubility of paclitaxel can be increased in the paclitaxel/lipiodol composition, which became stable for more than at least 200 days by adding a component that can inhibit paclitaxel precipitation.

1-35. (canceled)

36. A method of treating a tumor in a subject comprising administering to the subject a pharmaceutically effective amount of a composition comprising;
a) Lipiodol;
b) from 0.0001 mg to 13 mg of paclitaxel per 1 ml of said oily contrast medium; and
c) from 0.01 ml to 1 ml of an agent selected from the group consisting of alcohols, polyols, oils, lipids, poly lactide-co-glycolic acid and dimethylsulfoxide that prevent the formation of paclitaxel precipitation per 1 ml of said oily contrast medium.

37. The method of claim 36, wherein the Lipiodol is an iodized oil of iodine content ranging 30˜50% by weight.

38. The method of claim 37, wherein the Lipiodol is an iodized oil of iodine content ranging 35˜48% by weight.

39. The method of claim 36, wherein the oily contrast medium is iodized poppy seed oil with the iodine content of 35˜48% by weight.

40. The method of claim 36, further comprising 0.01˜1 ml of animal oil, vegetable oil or their mixture in 1 ml of the oily contrast medium.

41. The method of claim 40, wherein the animal oil is squalene.

42. The method of claim 40, wherein the vegetable oil is soybean oil.

43. The method of claim 36, wherein the viscosity is 40˜180 cP at room temperature.

44. The method of claim 36, wherein the tumor is a solid tumor.

45. The method of claim 44, wherein the solid tumor is hepatoma.

46. The method of claim 36, wherein the alcohol is selected from the group consisting of methanol, ethanol, propanol, isopropanol, butanol and fatty alcohols.

47. The method of claim 36, wherein the polyol is selected from the group consisting of ethylene glycol, propylene glycol and polyethyleneglycol.

48. The method of claim 36, wherein the oil is selected from the group consisting of triglycerides, diglyceride, monoglyceride, tocopherol and the mixtures thereof that can be extracted naturally from animal or vegetable oil.

49. The method of claim 36, wherein the lipid is selected from the group consisting of phospholipid, neutral lipid, cationic lipid, anionic lipid and fatty acid.

50. The method of claim 36, wherein the composition is administered directly into the tumor.
